(19) United States
(12) Patent Application Publication
(10) Pub. No.: US 2016/0287124 A1
(43) Pub. Date: Oct. 6, 2016

(54) ELECTROENCEPHALOGRAPHY (EEG) SIGNAL COLLECTING APPARATUS AND VEHICLE TERMINAL CONTROL SYSTEM

(71) Applicant: LEAUTO INTELLIGENT TECHNOLOGY (BEIJING) CO.LTD, Beijing (CN)

(72) Inventors: Yi HE, Beijing (CN); Minglu LIU, Beijing (CN)

(21) Appl. No.: 14/971,921

(22) Filed: Dec. 16, 2015

(30) Foreign Application Priority Data
Apr. 3, 2015 (CN) 201520202492.2

(51) Int. Cl. A61B 5/0478; A61B 5/18; A61B 5/00

(52) U.S. Cl. CPC A61B 5/0478; A61B 5/742; A61B 5/0022; A61B 5/18; A61B 5/7405

(57) ABSTRACT

The present disclosure is related to an electroencephalography signal collecting apparatus, comprising an electrode module, a TGAM module, a data outputting module, a power source module and a frame. The modules are disposed on the frame. The electrode module and the power outputting module are electrically connected to the TGAM module respectively. The electrode module transmits the collected electroencephalography signal to the TGAM module and is attached on the head of a wearer. The TGAM module is coupled to a vehicle apparatus outside of the electroencephalography signal collecting apparatus through the data outputting module, transmits the electroencephalography signal to a digital signal and outputs the digital signal to the vehicle apparatus. The vehicle intelligent terminal controlled through the electroencephalography signal is achieved and the operation of the driver is reduced. The intelligence and the convenience of the vehicle controlling operation is improved.

### CROSS REFERENCE TO RELATED APPLICATION

This present disclosure claims priority under 35 U.S.C. §119(a) to Patent Application No(s). 201520202492.2, filed in China on Apr. 3, 2015, the entire contents of which is hereby incorporated by reference.

### BACKGROUND

1. Technical Field

The present disclosure is related to vehicle apparatus technology field, and more particular to an electroencephalography signal collecting apparatus and a vehicle terminal control system.

2. Related Art

Vehicles have become indispensable transportation with the development of science and technology and the improvement of living standards. Intelligent driving and unmanned vehicles will also become the future trend of development.

It is hoped that the stimulation perceived by the human brain receptors is directly converted into an electrical signal to control the vehicle intelligent terminal in the intelligentized and highly efficient process of further improving the vehicle intelligent terminal. The control of the vehicle intelligent terminal is still suspended in the simple manual operation at present, for example, controlling the vehicle intelligent terminal through the body movement or the voice. However, the controlling operation through the body movement or the voice easily causes the driver's fatigue.

The approximate model is established by the modern neural network according to the structure, composition and basic unit of the cognitive basis of the human brain as well as the abstraction of human brain networks by combining mathematics, physics, information processing and other scientific methods. Although this concept of the bionic simulation can be infinitely close to the operational principle of the human neural network, a variety of complex algorithms and other scientific means are still required to approximate the human behavior. The concept not only has the computational complexity, but also is not yet able to be thinking like a human brain to sense in advance, analyze, judge and handle emergencies.

Thus, according to the aforementioned technical problems, an automatic controlling vehicle intelligent terminal and system based on the human electroencephalography signal are desirous.

### SUMMARY

Accordingly, the present disclosure provides an electroencephalography signal collecting apparatus to solve the technical problem of intelligent controlling of the vehicle terminal through the electroencephalography signal.

In order to solve the aforementioned technical problem, one embodiment of the present disclosure provides an electroencephalography signal collecting apparatus, comprising: an electrode module, a TGAM module, a data outputting module, a power source module and a frame. The electrode module, the TGAM module, the data outputting module and the power source module are disposed on the frame. Wherein, the electrode module and the power outputting module are electrically connected to the TGAM module respectively. The electrode module transmits the collected electroencephalography signal to the TGAM module. When the apparatus is worn, the electrode module attaches to the wearer's head. The TGAM module is coupled to a vehicle apparatus outside of the electroencephalography signal collecting apparatus through the data outputting module. The TGAM module converts the electroencephalography signal to a digital signal and outputs the digital signal to the vehicle apparatus.

The electrode module includes at least one first electrode and a second electrode; wherein the first electrode is a working electrode and the second electrode is a reference electrode of the electrode module. When the apparatus is worn, the first electrode and the second electrode are attached at different positions of the wearer's head respectively.

The frame is a spectacle-like frame; the first electrode and the second electrode are disposed at temples of the spectacle-like frame respectively.

The frame is an ear wrap frame; the first electrode and the second electrode are disposed at one side of the ear wrap near the ear respectively.

The frame is a circular resin frame; the first electrode and the second electrode are electrode patches, and are disposed at the inside of the circular resin frame respectively.

The data outputting module includes a data outputting port and a data line. One end of the data line is coupled to the data outputting port. The other end is coupled to the vehicle apparatus and the digital signal is outputted to the vehicle apparatus.

The frame further includes an accommodating groove. The data outputting port is disposed in the accommodating groove. The drawn back data outputting port is gathered in the accommodating groove and pulled out frame the accommodating groove.

The data outputting module includes a Bluetooth module or a WIFI module; the TGAM module outputs the digital signal to the vehicle apparatus by way of wireless communication.

In order to solve the aforementioned technical problem, one embodiment of the present disclosure provides a vehicle terminal control system, comprising a vehicle apparatus and the electroencephalography signal collecting apparatus disclosed in the present disclosure. The vehicle apparatus includes a CPU, a data receiving module matched with a data outputting module of the electroencephalography signal collecting apparatus and at least one peripheral module. Wherein, the CPU receives a digital signal outputted from the electroencephalography signal collecting apparatus through the data receiving module; the CPU converts the digital signal to a control signal and outputs the control signal to at least one peripheral module.

At least one peripheral module includes one or more of a navigation module, an audio playback module, a radio module, a camera module and a display module.

Compared with the prior art, the present disclosure may obtain the following technical effects:

1) The vehicle intelligent terminal controlled through the electroencephalography signal is achieved and the limb operation of the driver is reduced. The intelligence and the convenience of the vehicle controlling operation are improved.

2) The electrode module of the electroencephalography signal collecting apparatus is closely attached with the wearer's head and comfortable to be worn without oppressive feeling.

3) The reference electrode of the electrode module can adopt the electrode patch disposed at the ear. The reference voltage signal collected by the reference electrode is more stable and the influence caused by the shaking of the wearer's head is reduced.

4) The data outputting module of the electroencephalography signal collecting apparatus is disposed with the retractable data line for conveniently using the electroencephalography signal collecting apparatus and being easier for driver to carry.

Any product of the present disclosure will not necessarily need to meet all the technical effects described above.

### BRIEF DESCRIPTION OF THE DRAWINGS

The drawings herein provide a further understanding on the present disclosure, constitutes a part of this present disclosure. The exemplary embodiments of the present disclosure and the description thereof are used to explain the present disclosure. This does not constitute improper limitation on the present disclosure. In the drawings:

FIG. 1 is a block diagram of the electroencephalography signal collecting apparatus according to the embodiment of the present disclosure;

FIG. 2 is a principle schematic diagram of the electroencephalography signal collecting apparatus according to the embodiment of the present disclosure;

FIG. 3 is a schematic diagram of the electroencephalography signal collecting apparatus of FIG. 2 according to one embodiment of the present disclosure;

FIG. 4 is a schematic diagram of the electroencephalography signal collecting apparatus of FIG. 2 according to another embodiment of the present disclosure;

FIG. 5 is a schematic structure diagram of the electroencephalography signal collecting apparatus according to the embodiment of the present disclosure;

FIG. 6 is a block diagram of the vehicle terminal control system according to the embodiment of the present disclosure; and

FIG. 7 is a principle schematic diagram of the vehicle terminal control system connected to the electroencephalography signal collecting apparatus according to the embodiment of the present disclosure.

### DETAILED DESCRIPTION

The following description with reference to the accompanying drawings is provided to explain the exemplary embodiments of the present disclosure. The process of realization that how the present disclosure uses the technology to solve technical problems and reaches the technical effects can be fully understood and implemented accordingly.

### First Embodiment

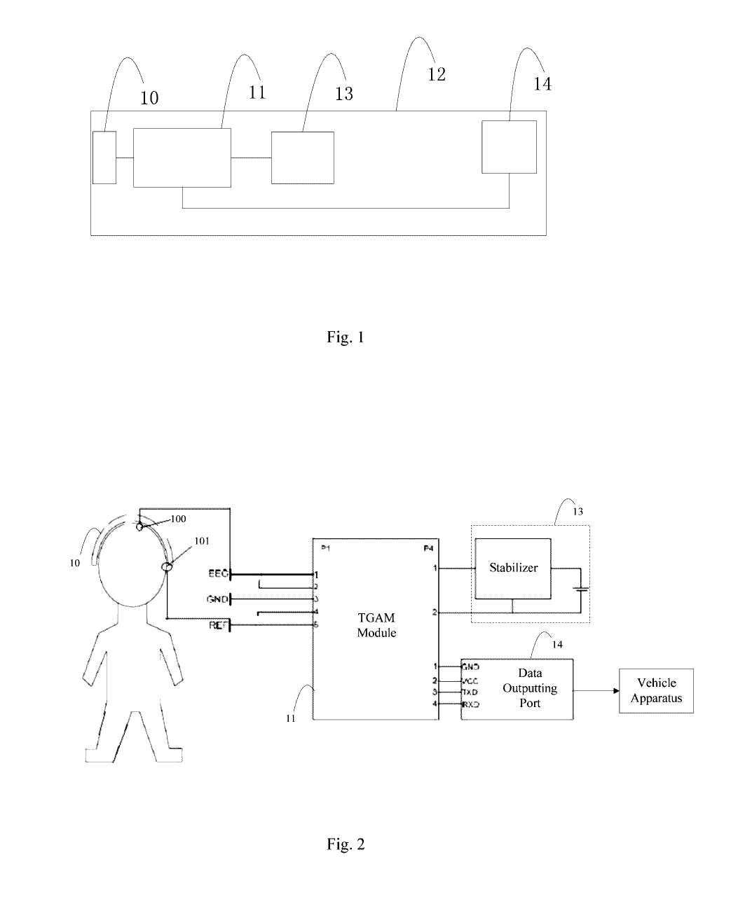

FIG. 1 is a block diagram of the electroencephalography signal collecting apparatus according to the embodiment of the present disclosure. The electroencephalography signal collecting apparatus includes an electrode module 10, a power source module 13, a TGAM (Think Gear Asic Module) module 11, a data outputting module 14 and a frame 12. The electrode module 10, the power source module 13, the data outputting module 14 and the TGAM module 11 are disposed on the frame 12. The electroencephalography signal collecting apparatus is worn on the user's head and collects the wearer's electroencephalography signal. Wherein, the power outputting module 13 is electrically connected to the TGAM module 11 through the internal conductive circuit of the frame 12 and supplies the electrical power to the TGAM module 11. After collecting the electroencephalography signal, the electrode module 10 is electrically connected to the TGAM module 11 through the internal conductive circuit of the frame 12 and transmits the collected electroencephalography signal to the TGAM module 11. When wearing the electroencephalography signal collecting apparatus, the electrode module needs to attach to the wearer's head in order to ensure the strength of the collected electroencephalography signal because the electroencephalography signal of the human is very weak.

The electrode module 10 collects the electroencephalography signal and transmits to the TGAM module 11 in real time. First, the TGAM module 11 filters the weaker electroencephalography signal in order to remove the signal interfering noise. For example, the interfering noise generated from wearer breathing or blinking in the electroencephalography signal is removed by filtering.

The data outputting module 14 is electrically connected to the TGAM module 11. The TGAM module 11 amplifies the filtered signal through its internal amplifying circuit such that the weaker electroencephalography signal is stronger and easily identified. The electroencephalography signal is transformed into the digital signal through the A/D (analog/digital) sampling. Finally, the digital signal is outputted to the vehicle apparatus through the data outputting module 14. The data transmitting method of the data outputting module can be by way of wire or wireless transmission. For example, the digital signal can be outputted to the vehicle apparatus through the Universal Asynchronous Receiver Transmitter (UART) communication interface, Bluetooth protocol or WIFI wireless network standard. The vehicle apparatus centralized controls for other vehicle intelligent terminals and analyzes the digital signal from the TGAM module 11 through the internal digital signal processor. The digital signal is transformed into the corresponding control command to control other vehicle apparatus terminals. The vehicle intelligent terminal controlled through the electroencephalography signal is achieved and the operation of the driver is reduced. The intelligence and the convenience of the vehicle controlling operation are improved.

### Second Embodiment

The electroencephalography signal collecting apparatus is shown in FIG. 2. Wherein, the electrode module 10 includes at least one first electrode 100 and a second electrode 101. When the apparatus is worn, the first electrode 100 and the second electrode 101 are attached to different positions of the wearer's head respectively. In one embodiment, the electrode module 10 includes a plurality of first electrodes 100 and the plurality of first electrodes 100 are centralizedly disposed.

Its working principle is that the first electrode 100 is a working electrode of the electrode module 10 and the second electrode 101 is a reference electrode of the electrode module 10. The TGAM module 11 induces the thinking of the human brain and the change of concern through the change of the potential difference between the first electrode 100 and the second electrode 101. Therefore, when the apparatus is worn, the position of the wearer's head which the second electrode 101 as a reference electrode is attached on should be fixed in order to prevent the reference voltage signal collected by the second electrode 101 from being changed. The first electrode 100 as a working electrode is used to collect the real time voltage signal of the wearer's brain. Therefore, the first electrode 100 should be attached on the wearer's head. The attaching position can be adjusted according to the strength of the collected real time voltage signal. The first electrode 100 is attached at the position which can collect the real time voltage signal having relatively stronger strength. The real time voltage signal and the reference voltage signal collected by the first electrode 100 and the second electrode 101 are sent to the TGAM module 11 respectively. As shown in FIG. 2, the first electrode 100 is coupled to the EEG pin of the TGAM module 11 and the second electrode 101 is coupled to the REF pin of the TGAM module 11. The TGAM module 11 induces the thinking of the wearer or the change of concern through the change of the potential difference between the received real time voltage signal and the reference voltage signal. The TGAM module 11 outputs the transformed digital signal to the vehicle apparatus through the data outputting module 14, as shown in FIG. 2.

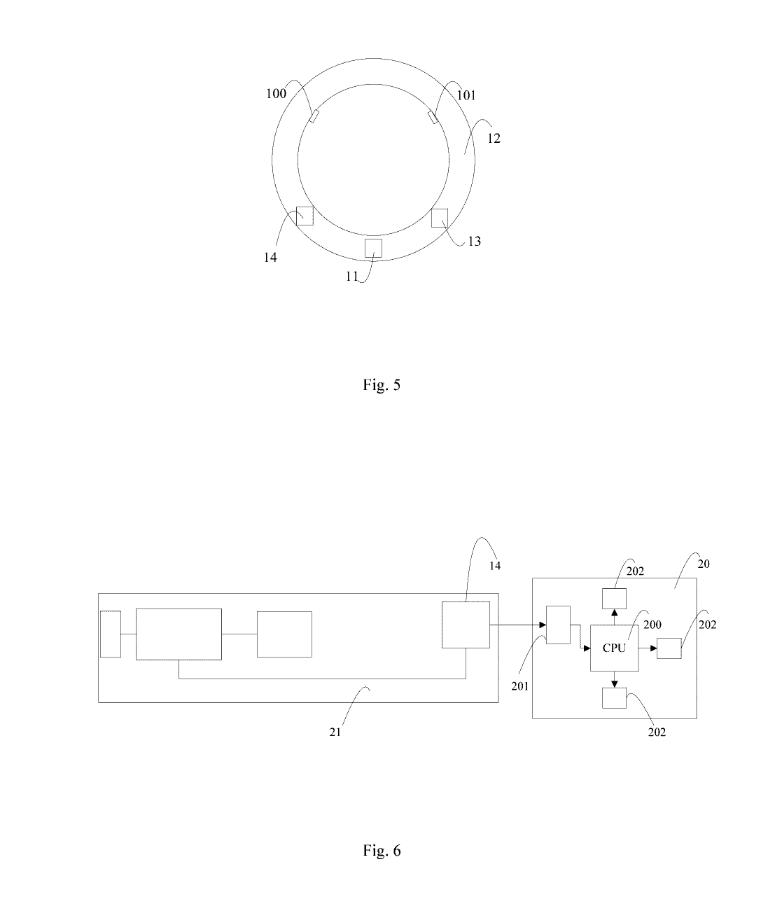

In a modification of the second embodiment, the frame 12 is a spectacle-like frame. The first electrode and the second electrode are disposed at the temples of the spectacle-like frame respectively. It is more convenient for the electroencephalography signal collecting apparatus to be worn by adopting the spectacle-like frame, which may use the ears for support, and which looks more fashionable. The first electrode 100 and the second electrode 101 can be electrode probes. The electrode probes adopt the flexible conductive material such that the first electrode 100 and the second electrode 101 can slide back and forth closely along the wearer's head in order to search out the best electroencephalography signal collecting position. Furthermore, using the flexible conductive material can make the wearer more comfortable and would not cause the oppression on the wearer's head. In one modification of the second embodiment, the frame 12 is an ear wrap frame as shown in FIG. 2. The first electrode and the second electrode are disposed in the war wrap at one side near the ear respectively. The second electrode disposed at the ear makes the reference voltage signal collected by the reference electrode more stable and reduces the influence generated by the shaking of the wearer's head. In another modification of the second embodiment, the frame 12 is a circular resin frame as shown in FIG. 5. The first electrode and the second electrode are electrode patches and disposed at the inside of the circular resin frame respectively. The electrodes of the electroencephalography signal collecting apparatus are disposed around the wearer's head and closely attached with the head.

### Third Embodiment

In one modification of the aforementioned embodiments, the data transmission between the TGAM module 11 and the vehicle apparatus are achieved through the data outputting module 14 by adopting the wireless transmission or the wire transmission.

When adopting the wireless transmission, the data outputting module 14 includes: a Bluetooth module or a WIFI module. The TGAM module 11 outputs the digital signal to the vehicle apparatus through the Bluetooth module or the WIFI module.

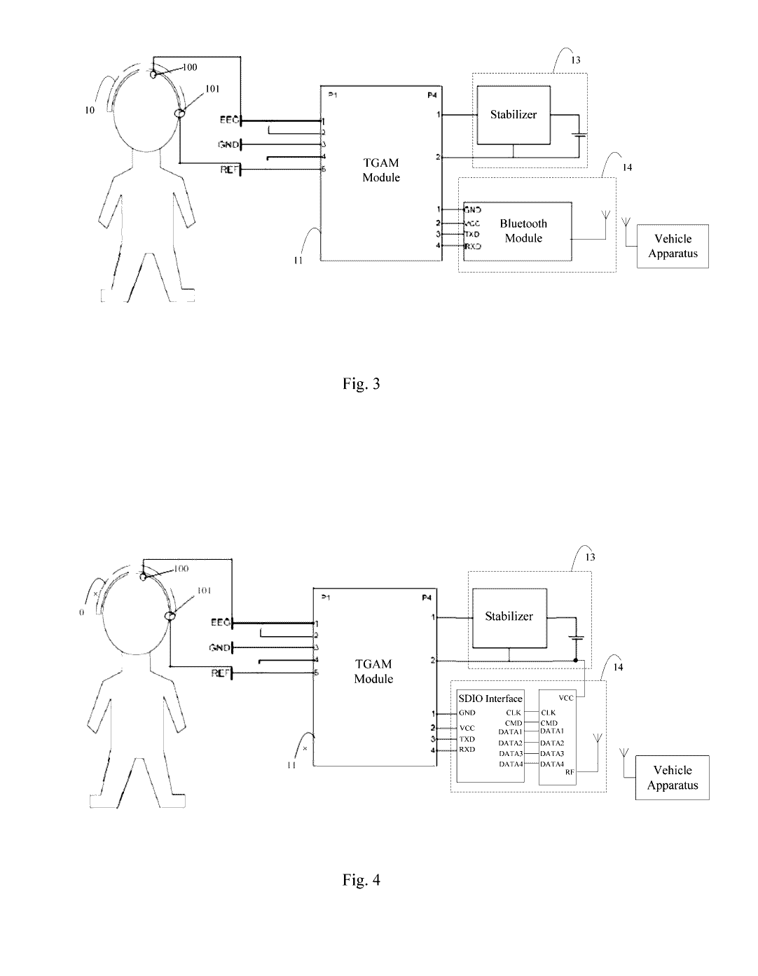

The electroencephalography signal collecting apparatus is shown in the FIG. 3. The data outputting module 14 is a Bluetooth module. The Bluetooth module outputs the digital signal to the vehicle apparatus through the Bluetooth protocol. For example, the Bluetooth module can be adopted by Bluetooth chip DA14580. The TGAM module 11 transmits the digital signal to the Bluetooth chip DA14580 through the UART communication interface. The Bluetooth chip DA14580 transmits the digital signal to the Bluetooth module of the vehicle apparatus through the Bluetooth protocol. The power consumption can be saved by using Bluetooth module for the data transmission. The driver will not be affected and it is easy for operation.

The electroencephalography signal collecting apparatus is shown in the FIG. 4. The data outputting module 14 is a WIFI module based on the Secure Digital Input/Output (SDIO) interface. The WIFI module based on the SDIO interface achieves the transformation of the digital signal between the SDIO interface and the wireless network. The WIFI module transmits the digital signal to the wireless network module of the vehicle apparatus through the WIFI wireless network standard. The data outputting module 14 further has a WIFI module with the UART interface and directly connects to the serial port of the TGAM module.

The data outputting module 14 includes a data outputting port and a data line when adopting the wire transmission. One end of the data line is coupled to the data outputting port. The other end is coupled to the data inputting port of the vehicle apparatus such that the communication is connected with the vehicle apparatus. The data outputting port and the data line can transmit the digital signal to the vehicle apparatus through the UART communication protocol. The volume of the electroencephalography signal collecting apparatus is smaller by adopting the wire transmission. The connection with the vehicle apparatus is more reliable and not interfered by external environment.

In one modification based on the third embodiments, the frame 12 further includes an accommodating groove. The data outputting port of the data outputting module is disposed in the accommodating groove. One end of the data line is coupled to the data outputting port. The data line is gathered in the accommodating groove and pulled out from the accommodating groove. The data line is an extendable data line. The data line is drawn back in the accommodating groove of the frame 12 when not used. When used, the data line is pulled from the accommodating groove of the frame 12 and adjusted to the suitable length. The length of the data line may be fixed. After finishing using the electroencephalography signal collecting apparatus, the data line will automatically retract to the accommodating groove of the frame 12 by pulling out a little bit. The data line with the extendable structure can reduce the usage space and be easily carried by users when the electroencephalography signal collecting apparatus is not used.

### Forth Embodiment

FIG. 6 is a block diagram of the vehicle terminal control system according to the forth embodiment of the present disclosure. The forth embodiment includes the vehicle apparatus 20 and any kind of the electroencephalography signal collecting apparatus 21 in aforementioned embodiment. The vehicle apparatus 20 includes a CPU 200, a data receiving module 201 matched with a data outputting module 14 of the electroencephalography signal collecting apparatus 21 and at least one peripheral module 202. Wherein, the CPU 200 receives a digital signal outputted from the data outputting module 14 of the electroencephalography signal collecting apparatus 21 through the data receiving module 201 and outputs the transformed control signal to at least one peripheral module 202. The data outputting module 14 of the electroencephalography signal collecting apparatus 21 includes a wire data outputting module based on the UART communication interface protocol and a wireless data outputting module based on the Bluetooth protocol or WIFI wireless network standard. Corresponsively, the data receiving module 201 of the vehicle apparatus 20 includes a wire data outputting module based on the UART communication interface protocol and a wireless data outputting module based on the Bluetooth protocol or WIFI wireless network standard.

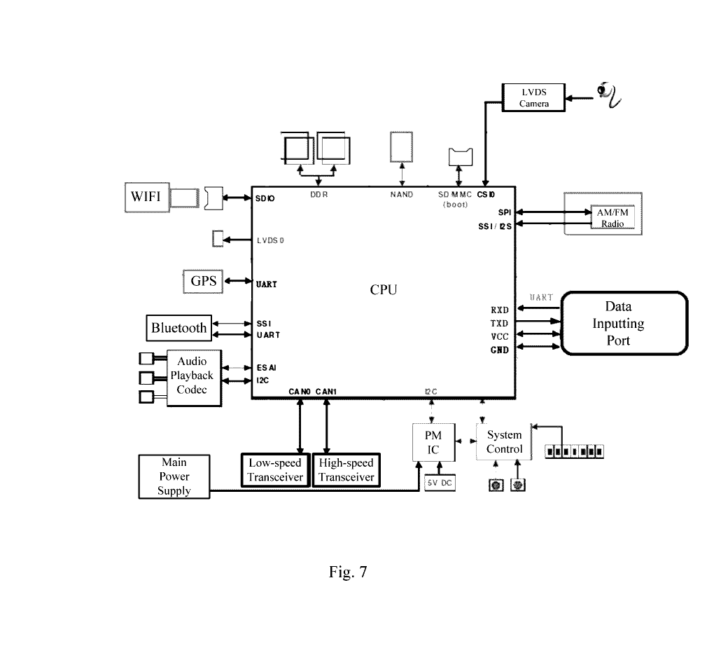

At least one peripheral module 202 includes one or more of a navigation module, an audio playback module, a radio module, a camera module and a display module. FIG. 7 is an internal principle schematic diagram of the vehicle terminal control system. The CPU connects to a plurality of peripheral module through the plurality of default communication port. The CPU computes and analyzes the digital signal analyzed by the digital signal processor through the default algorithm. The control commands in the computer language are generated. The CPU manages and controls each peripheral module respectively according to the flow of the operation system. For example, the CPU receives the control commands in the computer language and controls the GPS navigation module through the Universal Asynchronous Receiver/Transmitter (UART) interface protocol. Therefore, the function of automatically controlling the GPS navigation through the electroencephalography signal is achieved. The CPU receives the control commands in the computer language and controls the audio coding and decoding chip through the Intel-Integrated Circuit bus (I2C) and the Inter Integrated Circuit Sound Bus (I2S) interface protocol. Therefore, the function of automatically controlling the music through the electroencephalography signal is achieved. The CPU receives the control commands in the computer language and controls the radio module through the Intel-Integrated Circuit bus (I2C) and the Inter Integrated Circuit Sound Bus (I2S) interface protocol. Therefore, the function of automatically controlling the radio through the electroencephalography signal is achieved. The CPU receives the control commands in the computer language and controls the Sweep Panorama camera module through the Mobile Industry Processor Interface (MIPI) interface protocol. Therefore, the function of automatically controlling the panoramic parking through the electroencephalography signal is achieved. The CPU receives the control commands in the computer language and controls the Sweep Panorama camera module through the Intel-Integrated Circuit bus (I2C) and Low Voltage Differential Signal (LVDS) interface protocol. Therefore, the function of automatically controlling the switching of the car display through the electroencephalography signal is achieved.

For the realization of the aforementioned functions, the following procedure is used as an example. The vehicle driver wears the electroencephalography signal collecting apparatus and the electroencephalography signal collecting apparatus collects the driver's electroencephalography signal. When the driver's thinking focuses on the traffic in front of the vehicle, the driver's electroencephalography signal is more stable. When the driver wants to turn on or turn off the peripheral module in the vehicle, the concern of the driver's thinking will instantly change and the driver's electroencephalography signal will also change. The driver's electroencephalography signal is transmitted to the CPU of the vehicle apparatus by the electroencephalography signal collecting apparatus. When the instant change of the electroencephalography signal recognized by the CPU of the vehicle apparatus, the control commands in the computer language in accordance with the one or plurality of the peripheral modules are generated according to the default algorithm. Thus the function that any kind of the aforementioned peripheral modules controlled by the electroencephalography signal is achieved.

As used in the specification and claims, certain terms are used to refer to specific components. The person skilled in the art will appreciate that manufacturers may use different terms to refer to the same component. This specification and the claims do not differentiate the name as a way to distinguish between the components, but differentiate in the function of the component as a criterion to distinguish. As mentioned throughout the specification and claims, in which the “comprising” is an opening term, it should be interpreted as “including but not limited to.” “Approximately” means within an acceptable error range. Those skilled in the art solves the problem within a certain error range and substantially achieves the technical effect. In addition, “coupled” as used herein contains any direct and indirect electrical coupling means. Therefore, if the text describes a first device coupled to a second device, it means that the first device is directly electrically coupled to the second device or indirectly electrically coupled to the second device by other devices or coupled means. Subsequent description described in the present disclosure is for the implementation of the preferred embodiment, and then the description of the present disclosure is for explanation of the general principles, and is not intended to limit the scope of the present disclosure. The protection scope of the present disclosure is depended on the appended claims and their equivalents.

It is further noted that the term “comprising”, “including” or any other variation thereof, are intended to cover a non-exclusive inclusion, such that commodity or systems containing a series of factors not only include those elements, but also include other elements not explicitly listed, or also includes inherent elements for such commodity or systems. Without more constraints, elements defined by the statement “includes a” do not exclude the existence of additional identical elements in the element comprising the commodities or system.

The present disclosure is illustrated and described with reference to specific embodiments, but as previously described, it should be understood that the form of the present disclosure as disclosed herein is not limited. It should not be considered to exclude other embodiments, while available in various other combinations, modifications, and environments. It is capable to make changes by the techniques or knowledge in above teaching or related fields. Those skilled in the art will understand that many variations and modifications are readily attainable without departing from the spirit and scope thereof as defined by the appended claims and their legal equivalents.

1. An electroencephalography signal collecting apparatus, comprising:
an electrode module;
a TGAM module;
a data outputting module;
a power source module; and
a frame;
wherein the electrode module, the TGAM module, the data outputting module and the power source module are disposed on the frame;
wherein the electrode module and the power outputting module are electrically connected to the TGAM module respectively; the electrode module transmits the collected electroencephalography signal to the TGAM module; the worn electrode module is attached on the head of a wearer; the TGAM module is coupled to a vehicle apparatus outside of the electroencephalography signal collecting apparatus through the data outputting module; the TGAM module transmits the electroencephalography signal to a digital signal and outputs the digital signal to the vehicle apparatus.

2. The electroencephalography signal collecting apparatus of claim 1, wherein the electrode module comprises at least one first electrode and a second electrode; wherein the first electrode is a working electrode and the second electrode is a reference electrode of the electrode module; when the apparatus is worn, the first electrode and the second electrode are attached at different positions of the wearer's head respectively.

3. The electroencephalography signal collecting apparatus of claim 2, wherein the frame is a spectacle-like frame; the first electrode and the second electrode are disposed at the temples of the spectacle-like frame respectively.

4. The electroencephalography signal collecting apparatus of claim 2, wherein the frame is an ear wrap frame; the first electrode and the second electrode are disposed in the ear wrap at one side near the ear respectively.

5. The electroencephalography signal collecting apparatus of claim 2, wherein the frame is a circular resin frame; the first electrode and the second electrode are disposed at the inside of the circular resin frame respectively.

6. The electroencephalography signal collecting apparatus of claim 1, wherein the data outputting module comprises a data outputting port and a data line; wherein one end of the data line is coupled to the data outputting port; the other end is coupled to the vehicle apparatus and the digital signal is outputted to the vehicle apparatus.

7. The electroencephalography signal collecting apparatus of claim 6, wherein the frame further comprises an accommodating groove; the data outputting port is disposed in the accommodating groove; the data line is gathered in the accommodating groove and pulled out from the accommodating groove.

8. The electroencephalography signal collecting apparatus of claim 1, wherein the data outputting module comprises a Bluetooth module or a WIFI module; the TGAM module outputs the digital signal to the vehicle apparatus through the Bluetooth module or the WIFI module.

9. A vehicle terminal control system comprising a vehicle apparatus and a electroencephalography signal collecting apparatus according to claim 6, the vehicle apparatus comprising:
a CPU;
a data receiving module matched with a data outputting module of the electroencephalography signal collecting apparatus; and
at least one peripheral module;
wherein the CPU receives a digital signal outputted from the electroencephalography signal collecting apparatus through the data receiving module; the CPU transmits the digital signal to a control signal and outputs the control signal to at least one peripheral modules.

10. The vehicle terminal control system of claim 9, wherein at least one peripheral modules comprises one or more of a navigation module, an audio playback module, a radio module, a camera module and a display module.

11. A vehicle terminal control system comprising a vehicle apparatus and a electroencephalography signal collecting apparatus according to claim 8, the vehicle apparatus comprising:
a CPU;
a data receiving module matched with a data outputting module of the electroencephalography signal collecting apparatus; and
at least one peripheral module;
wherein the CPU receives a digital signal outputted from the electroencephalography signal collecting apparatus through the data receiving module; the CPU transmits the digital signal to a control signal and outputs the control signal to at least one peripheral modules.

12. The vehicle terminal control system of claim 11, wherein at least one peripheral modules comprises one or more of a navigation module, an audio playback module, a radio module, a camera module and a display module.
